(19) United States
(12) Patent Application Publication
(10) Pub. No.: US 2026/0235587 A1
(43) Pub. Date: Aug. 13, 2026

(54) COMPOSITIONS AND METHODS FOR STRUCTURAL AND MOLECULAR CHARACTERIZATION OF EXTRACELLULAR PARTICLES

(71) Applicant: Ohio State Innovation Foundation, Columbus, OH (US)

(72) Inventors: Eduardo REATEGUI, Columbus, OH (US); Truc NGUYEN KIM, Columbus, OH (US); Xilal RIMA, Columbus, OH (US)

(21) Appl. No.: 19/156,756

(22) PCT Filed: Feb. 15, 2024
(86) PCT No.: PCT/US2024/016033

§ 371 Date: Aug. 14, 2025

Related U.S. Application Data
(60) Provisional application No. 63/445,847, filed on Feb. 15, 2023.

(51) Int. Cl. G01N 33/53; G01N 15/14; G01N 15/1433; G01N 33/58; G06V 10/24; G06V 20/69

(52) U.S. Cl. CPC G01N 33/5308; G01N 15/1433; G01N 33/582; G01N 33/587; G06V 10/245; G06V 20/693; G06V 20/695; G01N 2015/1477; G01N 2015/1497

(57) ABSTRACT

Disclosed are systems and methods related to the multiparametric characterization of extracellular particles (EPs). The systems and methods can use a micropatterned substrate defining alignment features to correlating optical and non-optical images, thereby enabling the concurrent analysis of phenotypical and morphological features of EPs.

### CROSS-REFERENCE TO RELATED APPLICATIONS

This application claims the benefit of priority to U.S. Provisional Application No. 63/445,847, filed Feb. 15, 2023, which is hereby incorporated herein by reference in its entirety.

### STATEMENT OF GOVERNMENT SUPPORT

This invention was made with government support under Grant No. TR0002884 awarded by the National Institutes of Health. The government has certain rights in the invention.

### BACKGROUND

Extracellular particles (EPs) are highly heterogeneous with various particle subpopulations, including lipoproteins (LPs), extracellular vesicles (EVs), viruses, and non-vesicular extracellular particles. With the increasing interest in extracellular vesicles, which are cell-derived lipid nanoparticles that mediate aspects of interorgan communication and are biomolecular fountains for liquid biopsies, their isolation and subsequent characterization have been paramount. However, extracellular particles overlap in size and density; thus, their isolation is confounded by co-isolates of differing extracellular particle subpopulations. Therefore, the field has encouraged using orthogonal methods to ensure extracellular particle purity. Traditional molecular techniques for the characterization of extracellular particles require the bulk lysis of particles and dilute co-isolate profiles, promoting single-extracellular particle methods to investigate the degree of co-isolation or heterogeneity within extracellular particle subpopulations. Recently, single-extracellular vesicle detection utilizing optical and non-optical microscopic techniques has identified the co-isolation of extracellular vesicles and lipoproteins at the single-extracellular particle level, emphasizing the motivation to characterize extracellular particles at a single-extracellular particle resolution to comprehend co-isolation dynamics. However, both techniques have limitations. Super-resolution microscopy (SRM) has demonstrated potential in clarifying the heterogeneity of extracellular particles through fluorescence co-localization and subpopulation-based positive immunoselection. However, extracellular particles that downregulate the epitopes targeted by the fluorescent probes are excluded from the analyses thus producing false-negative biases. Furthermore, morphologies of the particles are obscured due to the diffraction limit. On the other hand, while electron microscopy (EM) has provided fundamental morphological data on extracellular particles and visualizes all extracellular particles without biases to antigens, phenotyping via immunogold labeling is hardly quantifiable due to the low efficiencies and limitations in multiplexing. Therefore, a technique that provides both the phenotyping ability of super-resolution microscopy techniques and the morphological quantification of electron microscopy techniques would further enhance the field and limit biases in quantification.

### SUMMARY

In accordance with the purposes of the disclosed materials, compounds, compositions, articles, devices, and methods, as embodied and broadly described herein, the disclosed subject matter relates to compositions and methods of making and using the compositions. In a specific aspect, disclosed herein are methods for multiparametric characterization of extracellular particles (EPs). In some aspects, the method includes:
contacting a sample comprising a population of EPs with a micropatterned substrate to adsorb a subpopulation of EPs on an imaging surface of the substrate, wherein the micropatterned substrate defines an alignment feature disposed thereon,
obtaining an optical image of the adsorbed and/or bound EPs with the alignment feature;
obtaining a non-optical image of the adsorbed and/or bound EPs with the alignment feature; and
correlating the optical image and the non-optical image using the alignment feature as a reference to form a consolidated feature set from the optical and non-optical images.

In some aspects, the method further includes determining structural and/or molecular properties of an individual EP from the consolidated feature set.

In some aspects, the micropatterned substrate comprises a transmission electron microscope (TEM) grid.

In some aspects, the micropatterned substrate comprises a functionalized nanolayer disposed on the imaging surface. In some aspects, the functionalized nanolayer comprises an extracellular particle binding region, wherein the EP binding region defines a geometry of the alignment feature. In some aspects, the functionalized nanolayer comprises a passivation region at least partially surrounding the EP binding region. In some aspects, the EP binding region of the functionalized nanolayer comprises an EP capture moiety coupled to a labeling group, wherein the EP capture moiety selectively binds the subpopulation of EPs, and wherein the labeling group is immobilized on the imaging surface. In some aspects, the EP capture moiety comprises an antibody, a polypeptide, and/or a nucleic acid. In some aspects, the antibody comprises anti-CD63, anti-CD9, anti-V5, conformation-specific antibodies, and/or as a user-defined antibody or antibody cocktail to target EPs In some aspects, the polypeptide comprises a recombinant protein such as ACE2, RGD peptide sequences, and/or electrostatic proteins, such as poly-L-lysine.

In some aspects, the labeling group comprises a streptavidin-functionalized gold nanoparticle immobilized on the imaging surface by adsorption. In some aspects, the passivation layer comprises a polyether, polyethylene glycol, and/or poly(L-lysine)-grafted-poly(ethylene glycol) (PLL-g-PEG).

In some aspects, the alignment feature is etched into the functionalized nanolayer.

In some aspects, the alignment feature comprises an asymmetric character.

In some aspects, a concentration of the extracellular particles in the sample is 1×10¹⁴ extracellular particles/mL or less, such as 5×10¹³ extracellular particles/mL or less, 2×10¹³ extracellular particles/mL or less, 5×10¹² extracellular particles/mL or less, 2×10¹² extracellular particles/mL or less, 5×10¹¹ extracellular particles/mL or less, 2×10¹¹ extracellular particles/mL or less, 5×10¹⁰ extracellular particles/mL or less, 2×10¹⁰ extracellular particles/mL or less, 5×10⁹ extracellular particles/mL or less, or 2×10⁹ extracellular particles/mL or less.

In some aspects, the EPs are tagged with a labeling group. In some aspects, the EPs are tagged with a fluorophore, dye, or fluorescent protein, such as a fluorescent-conjugated antibody and/or chromophore.

In some aspects, the step of correlating the optical and non-optical images includes forming a composite image by rotating and/or translating one or more of the optical and/or non-optical images to superimpose the alignment feature between the optical and non-optical images.

In some aspects, the optical image comprises a super-resolution microscopy (SRM) image. In some aspects, the SRM image comprises a total internal reflection fluorescence microscope (TIRFM) image.

In some aspects, the non-optical image comprises an electron microscope (EM) image. In some aspects, the EM image comprises a TEM image.

Also described herein are systems for the multiparametric characterization of extracellular particles (EPs). In various examples, the system includes:
a micropatterned substrate having an imaging surface and defining an alignment feature disposed thereon, wherein the micropatterned substrate is structured to adsorb a subpopulation of EPs on the imaging surface from a sample,
an optical imaging element positionable to collect optical images of adsorbed EP and the alignment feature;
a non-optical imaging element positionable to collect non-optical images of adsorbed and/or bound EPs and the alignment feature; and
a processor; and
a memory having instructions stored thereon, wherein execution of the instructions by the processor causes the processor to:
   receive optical and non-optical images showing the subpopulation of adsorbed and/or bound EPs; and
   orient the optical and non-optical images using the alignment feature to form a consolidated feature set from the optical and non-optical images.

In some aspects, the processor is further configured to determine structural and/or molecular properties of an individual EP from the consolidated feature set.

In some aspects, the micropatterned substrate comprises a transmission electron microscope (TEM) grid.

In some aspects, the micropatterned substrate comprises a functionalized nanolayer disposed on the imaging surface. In some aspects, the functionalized nanolayer comprises an extracellular particle binding region, wherein the EP binding region defines a geometry of the alignment feature. In some aspects, the functionalized nanolayer comprises a passivation region at least partially surrounding the EP binding region. In some aspects, the EP binding region of the functionalized nanolayer comprises an EP capture moiety coupled to a labeling group, wherein the EP capture moiety selectively binds the subpopulation of EPs, and wherein the labeling group is immobilized on the imaging surface. In some aspects, the EP capture moiety comprises an antibody, a polypeptide, and/or a nucleic acid. In some aspects, the antibody comprises anti-CD63, anti-CD9, anti-V5, conformation-specific antibodies, and/or as a user-defined antibody or antibody cocktail to target EPs In some aspects, the polypeptide comprises a recombinant protein such as ACE2, RGD peptide sequences, and/or electrostatic proteins, such as poly-L-lysine. In some aspects, the labeling group comprises a streptavidin-functionalized gold nanoparticle immobilized on the imaging surface by adsorption. In some aspects, the passivation layer comprises polyether, polyethylene glycol, and/or poly(L-lysine)-grafted-poly(ethylene glycol) (PLL-g-PEG).

In some aspects, the alignment feature is etched into the functionalized nanolayer. In some aspects, the alignment feature comprises an asymmetric character.

In some aspects, the EPs are tagged with a labeling group. In some aspects, the EPs are tagged with a fluorophore, dye, or fluorescent protein, such as a fluorescent-conjugated antibody and/or chromophore.

In some aspects, the processor is configured to generate a composite image of the optical and non-optical images by rotating and/or translating one or more of the images to superimpose the alignment feature in the optical and non-optical images.

In some aspects, the optical image comprises a super-resolution microscopy (SRM) image. In some aspects, the SRM image comprises a total internal reflection fluorescence microscope (TIRFM) image.

In some aspects, the non-optical image comprises an electron microscope (EM) image. In some aspects, the EM image comprises a TEM image.

Additional advantages of the disclosed subject matter will be set forth in part in the description that follows, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying Figures, which are incorporated in and constitute a part of this specification, illustrate several aspects of the invention and together with the description serve to explain the principles of the invention.

FIG. 1 shows a nano-positioning matrix technique to superimpose optical and non-optical images at the single-extracellular particle resolution according to one aspect. (a) Transmission electron microscopy (TEM) grids are functionalized with a non-biofouling polymer film, which is degraded in specific surface coordinates mediated by a digital-micromirror device (DMD)-based ultraviolet (UV) projection system. (b) Epifluorescence images of the photoetched micropatterns demonstrate the adsorption of protein species as asymmetrical numerals. (c) TEM images provide evidence for the binding of 50-nm streptavidin-functionalized gold nanoparticles (Strep-AuNPs) onto the micropatterns. (d) The micropatterns thus enable the subsequent functionalization with biotinylated antibodies for positive immunogold sorting. (e) The distinctive patterns visualized by fluorescent super-resolution microscopy (SRM) and electron microscopy (EM) enable the one-to-one mapping of optical and non-optical images via rotation and superimposition.

FIG. 2 shows the adaptability of the nano-positioning matrix technique to reduce colloidal stochasticity according to one aspect. (a) The electrostatic capture of extracellular particles (EPs) with a polycation-functionalized micropattern enables the non-biased immobilization of extracellular particles. The dotted squares are magnified in the proceeding insets. (b) The adsorption of streptavidin-functionalized gold nanoparticles onto the micropatterned surface is concentration-dependent, allowing for tunability in design (N=3 numerals, n=4 fields of view). The dotted squares are magnified in the insets. The calculations are provided in FIGS. 3-5. (c) Antibody-functionalized streptavidin-functionalized gold nanoparticles facilitate immunogold labeling of murine leukemia viruses (MLVs) onto the TEM grid mesh surface. The dotted outline represents the boundary of the micropattern. The three squares are magnified in the proceeding insets. (d) Total internal reflection fluorescence microscopy (TIRFM) and TEM are mapped one-to-one allowing the correlation of non-optical and optical images of single extracellular vesicles (EVs). The dotted outlines represent the boundary of the micropattern. The squares are magnified in the proceeding insets. On the magnified TEM image, the square is magnified as an inset and the circles define the perimeters of the extracellular vesicles. (e) The distribution of fluorescent (FL) and non-fluorescent (non-FL) particles are represented as a histogram-box-plot conjugate, demonstrating the similarity between non-fluorescent and fluorescent extracellular vesicles as measured in the TEM images (N=4 numerals, n=3 fields of view).

FIG. 3 shows the quantification of streptavidin-functionalized gold nanoparticle adsorption of the alignment number “9” pursuant to Example 1.

FIG. 4 shows the quantification of streptavidin-functionalized gold nanoparticle adsorption of the alignment number “7” pursuant to Example 1.

FIG. 5 shows the quantification of streptavidin-functionalized gold nanoparticle adsorption of the alignment number “6” pursuant to Example 1.

FIG. 6 shows single numeral streptavidin-functionalized gold nanoparticle micropatterns on individual square meshes. Single numeral micropatterns with Streptavidin-functionalized gold nanoparticles are adsorbed onto single square meshes on the transmission electron microscopy (TEM) grids. The square is magnified in the proceeding inset.

FIG. 7 shows Electrostatic immobilization of extracellular vesicles onto a micropattern. The electrostatic immobilization of extracellular vesicles (EVs) enables the non-biased capture of extracellular vesicles onto the mesh surface. The squares are magnified in panels a-i, where (a) illustrates the capture of extracellular vesicles onto the polycation surface with a classical “cup shape” morphology, (b) electron-dense extracellular vesicles without the classical “cup shape” morphology, (c) the polycation surface devoid of extracellular vesicles, (d) multicompartment extracellular vesicles, (e) protein aggregates, (f) positively stained extracellular vesicles with compromised membranes, (g) the boundary of the polycation surface, (h) a magnification of the square in (g) with minuscule extracellular particle structures, and (i) the mesh outside the functionalized micropattern region. The arrows indicate the location of the aforementioned phenomena.

FIG. 8 shows the quantification of extracellular vesicle size based on TEM images according to Example 1. (a) The whole extracellular vesicle population demonstrates a bimodal distribution. (b) The smallest distribution is normally distributed and corresponds to the minuscule extracellular particle structures. (c) The largest distribution is left-skewed with the majority of particles falling below the limit of detection of tunable resistive pulse sensing (TRPS).

FIG. 9 shows tunable resistive pulse sensing measurements. (a) The tunable resistive pulse sensing measurements of the bioreactor-generated extracellular vesicles do not contain the extracellular vesicle subpopulations measured in the TEM images. (b) The tunable resistive pulse sensing measurements of fluorescent extracellular vesicles are similar in size to the bioreactor-generated extracellular vesicles.

FIG. 10 shows a negative control for the capture of extracellular vesicles. Streptavidin-functionalized gold nanoparticles without antibody functionalization are unable to capture extracellular vesicles onto the micropatterned surface. The squares are magnified in panels a-d.

FIG. 11 shows the superimposition of fluorescent extracellular vesicles. (a) Total internal reflection fluorescence microscopy (TIRFM) illustrates the specific capture of extracellular vesicles within the micropatterned grid surface. The dotted outlines represent the boundary of the micropattern. (b) One-to-one mapping of extracellular vesicles from total internal reflection fluorescence microscopy images to TEM images reveals that a majority of extracellular vesicles are non-fluorescent (non-FL) and opposed to fluorescent (FL) (N=4 numerals, n=3 fields of view; the error bars indicate the standard error of the mean). (c) The fluorescence intensity of extracellular vesicles only correlates minimally with their measured diameter (N=4 numerals, n=3 fields of view). (d) The magnifications of the squares in FIG. 11, panel a are provided. The squares are magnified as an inset and the circles define the perimeters of extracellular vesicles.

### DETAILED DESCRIPTION

The compounds, compositions, articles, devices, and methods described herein can be understood more readily by reference to the following detailed description of specific aspects of the disclosed subject matter and the Examples and Figures.

Before the present compounds, compositions, articles, devices, and methods are disclosed and described it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

As used herein, the article “a,” “an,” and “the” means “at least one,” unless the context in which the article is used clearly indicates otherwise.

“Optional” or “optionally” means that the subsequently described event or circumstance may or may not occur, and that the description includes instances where said event or circumstance occurs and instances where it does not.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double-stranded polymer composed of nucleotide monomers. In some embodiments, the polynucleotide is composed of nucleotide monomers of generally greater than 100 nucleotides in length and up to about 8,000 or more nucleotides in length.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle-dependent or developmental stage-dependent manner, which may or may not also be tissue or cell-type specific. In some embodiments, a vector comprises one or more pol III promoters (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It is appreciated by those skilled in the art that the design of the expression vector can depend on such factors as the choice of the host cell to be transformed, the level of expression desired, etc.

The term “recombinant” refers to a human-manipulated nucleic acid (e.g. polynucleotide) or a copy or complement of a human-manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human-manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (ncbi.nlm.nih.gov). This algorithm involves first identifying high-scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence-specific manner.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

The term “about” as used herein when referring to a measurable value such as an amount, a percentage, and the like, is meant to encompass variations of ±20%, ±10%, ±5%, or ±1% from the measurable value.

A nucleic acid sequence is “heterologous” to a second nucleic acid sequence if it originates from a foreign species, or, if from the same species, is modified by human action from its original form. For example, a heterologous promoter (or heterologous 5′ untranslated region (5′UTR)) operably linked to a coding sequence refers to a coding sequence from a species different from that from which the promoter was derived, or, if from the same species, a coding sequence which is different from naturally occurring allelic variants.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen-combining sites and is still capable of cross-linking antigens.

As used herein, the term “antibody or antigen-binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv and the like, including hybrid fragments. Thus, fragments of the antibodies that retain the ability to bind their specific antigens are provided. For example, fragments of antibodies which maintain binding activity are included within the meaning of the term “antibody or antigen-binding fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen-binding fragment thereof” are conjugates of antibody fragments and antigen-binding proteins (single-chain antibodies). Also included within the meaning of “antibody or antigen-binding fragment thereof” are immunoglobulin single variable domains, such as for example a nanobody.

The fragments, whether attached to other sequences or not, can also include insertions, deletions, substitutions, or other selected modifications of particular regions or specific amino acid residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The terms “label” or “tag”, as used herein, refer to a composition capable of producing a detectable signal indicative of the presence of the target in an assay sample. Suitable labels include radioisotopes, nucleotide chromophores, enzymes, substrates, fluorescent molecules, chemiluminescent moieties, magnetic particles, bioluminescent moieties, and the like.

In various examples disclosed herein, the term “image”, refers to a two- or three-dimensional representation of a sample that contains spatial information. Images can includes, for example, any visual representation, such as a photo, a video frame, streaming video, as well as any electronic, digital, or mathematical analogue of a photo, video frame, or streaming video.

As used herein, the term “optical image” refers to an image obtained from an optical microscope. An “optical microscope” encompasses any optical microscope or light microscope that commonly uses visible light and a system of lenses to generate magnified images. Non-limiting examples of optical microscopy techniques that may be used with the present methods and systems include those configured for super-resolution microscopy, such as total internal reflection fluorescence microscopy (TIRFM), stimulated emission depletion (STED), reversible saturable optical linear fluorescence transitions (RESLOFT), ground state depletion (GSD), saturated structured illumination microscopy (SSIM), fluorescence photoactivation localization microscopy (FPALM), photoactivation localization microscopy (PALM), and stochastic optical reconstruction microscopy (STORM).

As used herein, the term “non-optical image” refers to an image obtained from an non-optical microscope. “Non-optical microscopes” refer to any device used to produce magnified images which do not rely on visible light or optical lenses. Non-limiting examples of non-optical imaging techniques include, but are not limited to, transmission electron microscopy (TEM), scanning electron microscopy (SEM), atomic force microscopy (AFM) and scanning tunneling microscopy (STM), raman spectroscopy, surface-enhanced raman spectroscopy (SERS) and the like.

The term “extracellular particle” broadly refers to the collection of lipoproteins (LPs), extracellular vesicles (EVs), viruses, outer membrane vesicles (OMVs) and non-vesicular extracellular particles.

As used here, the term “population of extracellular particles” refers to a plurality of extracellular particles having distinct characteristics. The term “subpopulation” when used in reference to a population of extracellular particles, refers to less than all extracellular particles from the population of particles. For example, the term subpopulation can refer to extracellular particles having a shared characteristic or trait.

Reference will now be made in detail to specific aspects of the disclosed materials, compounds, compositions, articles, and methods, examples of which are illustrated in the accompanying Examples and Figures.

### Methods

Disclosed herein are methods for multiparametric characterization of extracellular particles (EPs).

In some aspects, the method includes:
contacting a sample comprising a population of EPs with a micropatterned substrate to adsorb a subpopulation of EPs on an imaging surface of the substrate, wherein the micropatterned substrate defines an alignment feature disposed thereon,
obtaining an optical image of the adsorbed and/or bound EPs with the alignment feature;
obtaining a non-optical image of the adsorbed and/or bound EPs with the alignment feature; and
correlating the optical image and the non-optical image using the alignment feature as a reference to form a consolidated feature set from the optical and non-optical images.

In the present disclosure, the terms “micropatterned surface or substrate” generally refer to a surface or substrate that has at least one surface which has an intended plurality of features that define a profile characterized by raised portions and recessed portions which are raised or recessed relative to a datum surface. The features of the micropatterned substrate (e.g., the alignment feature) are sufficiently identifiable such that a distinguishable profile can be observed in both the optical and non-optical images. Features on the micropatterned substrate can form an alignment feature (e.g., a plurality of alignment features), which can be used as a reference point to correlate spatial dimensions between the optical and non-optical images.

In various examples, the micropatterned substrate comprises a functionalized nanolayer disposed on the imaging surface. As used throughout the present application, the term “functionalized” is to be understood, as the formation of a chemical bond, such as a covalent, coordinative, hydrogen bond, ionic, or dispersive (van-der-Waals) bond between a structure and a functional group. Similarly, the term “functionalized nanolayer” refers to a layer having a thickness less than 1 micron that is formed by the surface functionalization of a substrate (e.g., the micropatterned substrate) with a desired functional group.

The thickness of the functional nanolayer can affect the ability of compounds to chemically bind/adsorb thereto. In some examples, the thickness of the functionalized nanolayer is from about 1 nm to about 1 μm (e.g., about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm), inclusive of all ranges and subranges therebetween.

The alignment feature can include any distinguishable shape, character, or pattern, that is sufficiently identifiable in both the optical and non-optical images. In some aspects, the alignment feature is an asymmetric character. In various examples, the alignment feature is formed into a functionalized nanolayer based on a spatial affinity of the EP to the functionalized nanolayer.

In some aspects, the functionalized nanolayer includes an extracellular particle binding region, wherein the EP binding region defines a geometry of the alignment feature. In some aspects, the functionalized nanolayer comprises a passivation region at least partially surrounding the EP binding region. In some aspects, the EP binding region of the functionalized nanolayer comprises an EP capture moiety coupled to a labeling group, wherein the EP capture moiety selectively binds the subpopulation of EPs, and wherein the labeling group is immobilized on the imaging surface. The term “selectively binds” means the EP capture moiety preferentially binds to a target epitope on the EP compared to other binding groups. In some aspects, the EP capture moiety comprises an antibody, a polypeptide, a nucleic acid, and/or an aptamer. In some aspects, the antibody comprises anti-CD63, anti-CD9, and/or anti-V5, conformation-specific antibodies, and/or as a user-defined antibody or antibody cocktail to target EPs. Specific examples of some suitable EP capture moieties include those shown below in Table 1. In some aspects, the polypeptide includes a recombinant protein such as ACE2, RGD peptide sequences, and/or electrostatic proteins or polypeptides, such as salts of poly-L-ornithine, poly-L-arginine, poly-L-lysine, poly-D-lysine, polyallylamine and polyethyleneimine.

The EP binding region of the functionalized nanolayer can include a labeling group, which can be useful for increasing the visibility of EPs bound to the EP capture moiety. For example, the labeling group can be a polypeptide, a nucleic acid, and/or an aptamer, such as a luminescent, fluorescent, fluorogenic, chromogenic, magnetic, radioactive or other type of detectable label. In some aspects, the labeling group includes a biotin moiety, streptavidin moiety, bead, resin, a solid support, or a combination thereof. In some aspects, the labeling group includes a fluorophore, dye, or fluorescent protein, such as a fluorescent-conjugated antibody and/or chromophore.

A wide variety of different types of fluorophores are readily available and applicable to the disclosed methods and include fluorescein, or rhodamine based dyes, cyanine dyes and the like. A variety of such dyes are commercially available and include the Cy dyes available from GE Healthcare (Piscataway, N.J.), such as Cy3, Cy5, and the like, or the Alexa® family of dyes available from Invitrogen/Molecular Probes (Carlsbad, Calif.), such as Alexa 488, 500, 514, 532, 546, 555, 568, 594, 610, 633, 647, 660, 680, 700, and 750. It is appreciated that fluorophores may be present as individual fluorophores or they may be present in interactive pairs or groups, e.g., as fluorescent resonant energy transfer (FRET) pairs.

Other labeling strategies may employ inorganic materials as labeling groups, such as fluorescent or luminescent nanoparticles, e.g. nanocrystals, i.e. Quantum Dots, that possess inherent fluorescent capabilities due to their semiconductor make up and size in the nanoscale regime (See, e.g., U.S. Pat. Nos. 6,861,155, 6,699,723, 7,235,361). By way of example, nanocrystal materials are generally commercially available from, e.g., Invitrogen, Inc., (Carlsbad Calif.).

The substrate can, in some regions, be functionalized with a passivation layer to prevent or substantially inhibit adsorption or binding of EPs compared to other areas on the substrate (e.g., the EP binding region). In some aspects, the passivation layer prevents or inhibits adsorption or binding of EPs by 50% or more (e.g., 55% or more, 60% or more, 65% or more, 70% or more, 75% or more, 80% or more, 85% or more, 90% or more, 95% or more, 97% or more, or 99% or more) compared to other areas on the substrate. In some aspects, the passivation layer comprises a polyether, polyethylene glycol, and/or poly(L-lysine)-grafted-poly(ethylene glycol) (PLL-g-PEG).

In some aspects, the method further includes determining structural and/or molecular properties, such as EP phenotype and morphology, of an individual EP from the consolidated feature set.

In some aspects, the micropatterned substrate comprises a transmission electron microscope (TEM) grid. TEM grids can be formed from a variety of materials. Non-limiting examples of grid materials include, for example, Formvar/Carbon 75 mesh, Copper (Ted Pella, cat #01802-F), Carbon Type-B, 200 mesh TH, Nickel (Ted Pella, cat #01808N), Carbon Type-B, 300 mesh, Nickel (Ted Pella, cat #01813N).

The sample used according to the present method can include, for example, a biological or non-biological fluid. In various aspects, the sample is obtained from a mammalian subject. For example, the sample can be obtained from a human. In some aspects, the sample includes blood, urine, semen, milk, sputum, mucus, a buccal swab, a vaginal swab, a rectal swab, an aspirate, a needle biopsy, a section of tissue obtained for example by surgery or autopsy, plasma, serum, spinal fluid, lymph fluid, the external secretions of the skin, respiratory, intestinal, and genitourinary tracts, tears, saliva, tumors, or organs that include EPs.

In some aspects, a concentration of the extracellular particles in the sample is 1×10¹⁴ extracellular particles/mL or less, such as 5×10¹³ extracellular particles/mL or less, 2×10¹³ extracellular particles/mL or less, 5×10¹² extracellular particles/mL or less, 2×10¹² extracellular particles/mL or less, 5×10¹¹ extracellular particles/mL or less, 2×10¹¹ extracellular particles/mL or less, 5×10¹⁰ extracellular particles/mL or less, 2×10¹⁰ extracellular particles/mL or less, 5×10⁹ extracellular particles/mL or less, or 2×10⁹ extracellular particles/mL or less. In some aspects, the concentration of extracellular particles in the sample is 1×10⁹ extracellular particles/mL or more, such as 2×10⁹ extracellular particles/mL or more, 5×10⁹ extracellular particles/mL or more, 2×10¹⁰ extracellular particles/mL or more, 5×10¹⁰ extracellular particles/mL or more, 2×10¹¹ extracellular particles/mL or more, 5×10¹¹ extracellular particles/mL or more, 2×10¹² extracellular particles/mL or more, 5×10¹² extracellular particles/mL or more, 2×10¹³ extracellular particles/mL or more, 5×10¹³ extracellular particles/mL or more, or 1×10¹⁴ extracellular particles/mL or more. In some aspects, the concentration of extracellular particles in the sample ranges from any of the lower values to any of the higher values.

In some aspects, the step of correlating the optical and non-optical images includes forming a composite image by rotating and/or translating one or more of the optical and/or non-optical images to superimpose the alignment feature between the optical and non-optical images.

In some aspects, the optical image comprises a super-resolution microscopy (SRM) image. Examples of suitable optical imaging techniques include, for example, total internal reflection fluorescence microscopy (TIRFM), stimulated emission depletion (STED), reversible saturable optical linear fluorescence transitions (RESLOFT), ground state depletion (GSD), saturated structured illumination microscopy (SSIM), fluorescence photoactivation localization microscopy (FPALM), photoactivation localization microscopy (PALM), and stochastic optical reconstruction microscopy (STORM). In some aspects, the SRM image comprises a total internal reflection fluorescence microscope (TIRFM) image.

In some aspects, the non-optical image comprises an electron microscope (EM) image. Typically, EMs are able to view detail at the atomic level with sub-nanometer resolution (e.g., 0.1 nm resolution). In a TEM, electrons are typically transmitted through a thinly sliced specimen and typically form an image on a fluorescent screen or photographic plate. Those areas of the sample that are denser transmit fewer electrons and therefore appear darker in the resulting image. In some aspects, the EM image comprises a TEM image.

### Systems

Also described herein are systems for the multiparametric characterization of extracellular particles (EPs). In various examples, the system includes:
a micropatterned substrate having an imaging surface and defining an alignment feature disposed thereon, wherein the micropatterned substrate is structured to adsorb a subpopulation of EPs on the imaging surface from a sample,
an optical imaging element positionable to collect optical images of adsorbed EP and the alignment feature;
a non-optical imaging element positionable to collect non-optical images of adsorbed and/or bound EPs and the alignment feature; and
a processor; and
a memory having instructions stored thereon, wherein execution of the instructions by the processor causes the processor to:
   receive optical and non-optical images showing the subpopulation of adsorbed and/or bound EPs; and
   orient the optical and non-optical images using the alignment feature to form a consolidated feature set from the optical and non-optical images.

In some aspects, the processor is further configured to determine structural and/or molecular properties, such as EP phenotype and morphology, of an individual EP from the consolidated feature set.

In some aspects, the micropatterned substrate comprises a transmission electron microscope (TEM) grid. Non-limiting examples of grid materials include, for example, Formvar/Carbon 75 mesh, Copper (Ted Pella, cat #01802-F), Carbon Type-B, 200 mesh TH, Nickel (Ted Pella, cat #01808N), Carbon Type-B, 300 mesh, Nickel (Ted Pella, cat #01813N)

In some aspects, the micropatterned substrate comprises a functionalized nanolayer disposed on the imaging surface. In various examples, the thickness of the functionalized nanolayer is from about 1 nm to about 1 μm (e.g., about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm), and is inclusive of all ranges and subranges therebetween.

In some aspects, the functionalized nanolayer includes an extracellular particle binding region, wherein the EP binding region defines a geometry of the alignment feature. In some aspects, the functionalized nanolayer comprises a passivation region at least partially surrounding the EP binding region. In some aspects, the EP binding region of the functionalized nanolayer comprises an EP capture moiety coupled to a labeling group, wherein the EP capture moiety selectively binds the subpopulation of EPs, and wherein the labeling group is immobilized on the imaging surface. In some aspects, the EP capture moiety comprises an antibody, a polypeptide, a nucleic acid, and/or an aptamer. In some aspects, the antibody comprises anti-CD63, anti-CD9, and/or anti-V5, conformation-specific antibodies, and/or as a user-defined antibody or antibody cocktail to target EPs. Specific examples of some suitable EP capture moieties include those shown below in Table 1. In some aspects, the polypeptide includes a recombinant protein such as ACE2, RGD peptide sequences, and/or electrostatic proteins or polypeptides, such as salts of poly-L-ornithine, poly-L-arginine, poly-L-lysine, poly-D-lysine, polyallylamine and polyethyleneimine.

In some aspects, the alignment feature is etched into the functionalized nanolayer. In some aspects, the alignment feature comprises an asymmetric character.

In some aspects, the population of EPs is configured to emit a fluorescent signal. The EP binding region of the functionalized nanolayer can include a labeling group, which can be useful for increasing the visibility of EPs bound to the EP capture moiety. For example, the labeling group can be a polypeptide, a nucleic acid, and/or an aptamer, such as a luminescent, fluorescent, fluorogenic, chromogenic, magnetic, radioactive or other type of detectable label. In some aspects, the labeling group includes a biotin moiety, streptavidin moiety, bead, resin, a solid support, or a combination thereof. In some aspects, the labeling group includes a fluorophore, dye, or fluorescent protein, such as a fluorescent-conjugated antibody and/or chromophore.

In some aspects, the processor is configured to generate a composite image of the optical and non-optical images by rotating and/or translating one or more of the images to superimpose the alignment feature in the optical and non-optical images.

In some aspects, the optical image comprises a super-resolution microscopy (SRM) image. Examples of suitable optical imaging techniques include, for example, total internal reflection fluorescence microscopy (TIRFM), stimulated emission depletion (STED), reversible saturable optical linear fluorescence transitions (RESLOFT), ground state depletion (GSD), saturated structured illumination microscopy (SSIM), fluorescence photoactivation localization microscopy (FPALM), photoactivation localization microscopy (PALM), and stochastic optical reconstruction microscopy (STORM). In some aspects, the SRM image comprises a total internal reflection fluorescence microscope (TIRFM) image. In some aspects, the SRM image comprises a total internal reflection fluorescence microscope (TIRFM) image.

In some aspects, the non-optical image comprises an electron microscope (EM) image. In some aspects, the EM image comprises a TEM image.

The processor of the described system, for processing information associated with the process(es) or method(s) described herein, maybe, for example, any computer processor is known in the art capable of performing calculations and directing functions for interpreting and/or performing input, output, calculation, and display of data in accordance with the disclosed methods. The processor may comprise any type of processing unit, such as typical computer processor(s), controller(s), microcontroller(s), microprocessor s), and/or programmable logic controllers (PLCs). The information to be processed by the processor may include, for example, information contained in analog or digital signals and/or translated signals and/or information contained in a data storage. Processing of the information may involve, for example, performing calculations on received signals such as, but not limited to, vector analysis, picture identification, pattern recognition, frequency analysis/Fourier transforms, numerical computations, machine learning, or, as described herein, applying predetermined or recursively fit correlative algorithms to received sensor data. In some embodiments, the system comprises more than one processor, and the reference herein to “processor” includes reference to multiple processors and vice versa. In various examples, the processor is configured to receive user-generated instructions (e.g., the target location and/or the target orientation).

In an embodiment, the processor is in communication with both the imaging element and power source. In another embodiment, the processor may also be in communication with data storage(s), and, optionally, display(s). The components of the system, such as the imaging element, power supply, sensors, computing device(s), processor(s), feedback controller(s), data storage(s), and/or display(s), and any other components of system, device or computing device, may communicate using any electronic wired or wireless means or protocols for communication known in the art, including but not limited to Ethernet™, Bluetooth™, WiFi™, infrared, near-field communications (NFC), radio-frequency identification (RFID), WiMAX™ (fixed or mobile), cellular communications protocols such as GSM, EDGE, GPRS, CDMA, EMTS, LTE, LTE-A, IMS, and any other cellular communications protocols including, but not limited to, up to and including 5G protocols as established under the 3 GPP, for example, and any other communications protocols suitable for the method(s) and system(s) described herein, including any proprietary protocols. Components of the system may exist on the same network or on separate networks, and the network(s) may include any type of network suitable for the system(s) and method(s) described herein, including but not limited to wired or wireless personal area networks (PANs), local area networks (LANs), mesh or ad hoc networks, wide area networks (WANs), metropolitan area networks (MANs), virtual private networks (VPNs), and any other suitable network type, as well as any suitable network configuration or topology (e.g., token ring, star, bus, mesh, tree, etc.). The presently described system(s) further includes any components necessary to effect the communication and/or network type employed, such as wireless or wired routers and access points.

The presently described methods and systems may be implemented on a secure network to which access may be limited to authorized users by any known means and which may be protected by known security measures, such as by the use of firewalls. In some embodiments, authentication may be required before granting access to authorized users, such as where autologous cell manufacturing and/or patient data is involved and/or where compliance with government regulations is mandated (such as Title 21 of the U.S. Code of Federal Regulations). Such authentication may be implemented for any one or more of the system components, such as for access to a computing device or machine housing the processor, access to data storage, access to a database of the data storage, access to any of the sensors or sensor readings of the device or imaging element, access to a graphical user interface (GUI) of the system, access to the device or imaging element, etc. Security of the presently described methods and systems may be further provided for by encrypting communications among system components by any means or protocols known to persons skilled in the art, such as Internet Protocol Security (IPSec), Transport Layer Security (TLS), Secure Sockets Layer (SSL), etc., in order to reduce the potential for the tampering with or corruption of the preprogrammed correlative algorithm(s).

The presently described system may also include a data storage for storing information associated with the described methods. The data storage may include, for example, various types of local or remote memory devices such as a hard disk or hard drive (of any type, including electromechanical magnetic disks and solid-state disks), a memory chip, including, e.g., random-access memory (RAM) and/or read-only memory (ROM), flash memory, optical memory such as CD(s) and DVD(s), floppy disks, and any other form of optical, physical, electronic, and/or magnetic memory devices in or on which information may be stored. The data storage may comprise non-volatile memory. In some embodiments, the data storage may only be accessed via secure data transfer, which may be accomplished using one or more known server platforms and security protocols. The information to be stored in the data storage may comprise, for example, one or more predetermined algorithms for correlating sensor inputs to various outputs and records of such predicted determinations along with associated actions (such as feedings) and/or associated timestamps, unique identifiers, authorized users and associated authentication information for use in authenticating users for authorized access to the data storage or any other system component, and any other pertinent information. In operation, the data storage is in communication with the processor.

The system may also include a display (which may be co-located with the processor, e.g., where the processor and display are part of a computer or server used for carrying out the method steps described herein) for visually presenting information associated with the described methods. The display may comprise, for example, a computer monitor (e.g., LCD, a CRT monitor, a projection (e.g., heads-up display (HUD) laser), etc. In some embodiments, the visual display may comprise, for example, that of a mobile device such as a tablet computer, cellular phone, smartphone, personal digital assistant (PDA), personal computer (PC), laptop computer, augmented reality display (e.g., Google™ Glass™ or Microsoft™ HoloLens™), etc. The information presented on the display may include any other information collected in the course of carrying out the methods described herein, prompts for information entry associated with one or more steps of the described methods, and/or any predetermined formulae or algorithms, as previously described. The display may also be capable of receiving input (such as, e.g., where the display includes a touch-screen and is capable of receiving touch input and accordingly transmitting information to the processor).

### EXAMPLES

The following examples are set forth below to illustrate the methods, compositions, and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: Limiting Brownian Motion to Enhance Immunogold Phenotyping and Superimpose Optical and Non-Optical Single-Extracellular Particles Analyses

Introduction. Optical and non-optical techniques propelled the field of single extracellular particle (EP) research through phenotypic and morphological analyses, revealing the similarities, differences, and co-isolation of extracellular particle subpopulations. Overcoming the challenges of optical and non-optical techniques motivates the use of orthogonal techniques while analyzing extracellular particles (EPs), which require varying concentrations and preparations.

Described in this example is a nano-positioning matrix (NPMx) technique, which is capable of performing orthogonal measurements at the single-extracellular particle resolution via superimposing micropatterns acquired through non-optical and optical microscopy. Micropatterning on transmission electron microscopy (TEM) grids through ultraviolet (UV)-induced degradation of a non-biofouling polymer brush enables the precise functionalization of the mesh surface in traceable coordinates. The tunable design provides electrostatic entrapment of extracellular particles for a non-biased capture of the entire subpopulation and facile immunogold labeling by limiting Brownian motion via tethering antibody-functionalized gold nanoparticles (AuNPs) onto the mesh surface. The distinctive micropatterns enabled the one-to-one mapping of super-resolution microscopy images to electron microscopy images to correlate fluorescence signals to extracellular vesicles captured on the mesh surface. The engineered surface thus allows for a systematic technique that enhances electron microscopy efficiencies, improves immunogold labeling, and provides a framework for an orthogonal measurement of the same extracellular particle.

### Materials and Methods

Fluorescent Extracellular Vesicle collection. Gli36 cells were transfected with PalmtdTomato [18,19] and were maintained in Dulbecco's Modified Eagle Medium (DMEM) supplemented with 10% (v/v) fetal bovine serum (FBS) and 1% (v/v) penicillin-streptomycin (PS). Once 70% confluence was achieved, the cells were washed with phosphate-buffered saline (PBS) and given serum-free DMEM for 48 hours, after which the supernatant was collected and centrifuged at 2,000×g for 10 min to remove cell debris and large extracellular vesicles. The supernatant was then purified via size-exclusion chromatography (SEC) with a 35 nm qEV original column by pooling extracellular vesicle-rich fractions 1~4 (Izon Science), according to the manufacturer's instructions.

Bioreactor extracellular vesicle collection. As previously described, U373 cells were cultured in a CELLine AD 1000 adherent bioreactor flask (Wheaton) [20,21]. Briefly, 25×10⁶ cells were inoculated in 15 mL of complete DMEM with 10% FBS and 1% PS in the lower cell chamber with ~750 mL of the same media added to the upper media chamber. The bioreactor was then gradually adapted from 10% FBS supplementation to 10% chemically-defined CDM-HD (Fibercell) serum replacement over four weeks to avoid exogenous FBS extracellular vesicle contamination. Extracellular vesicles were collected in ~15 mL of conditioned media three times per week from the cell chamber, and the media chamber was refreshed once weekly. The 15 mL of conditioned media was centrifuged at 2,000×g for 10 min to remove suspended cells and large debris, and the resulting supernatant was concentrated using a 100 kDa MWCO Vivaspin 20 filter (Sartorius) at 3,000×g to a volume of ~4 ml. This concentrated media was centrifuged at 10,000×g for 30 min to remove large extracellular vesicles, and the remaining supernatant was further concentrated using a 100 kDa Vivaspin 500 filter (Sartorius). The crude small extracellular vesicle suspension was then purified with SEC as previously described.

Tunable resistive pulse sensing (TRPS). Tunable resistive pulse sensing (qNano Gold, Izon Science) was utilized to measure the concentration and size of extracellular vesicles using a stretchable NP200 membrane. A pressure of 0.5 kPa and voltage of 0.38 V were maintained throughout the measurements. The measurements were calibrated with known standard polystyrene nanoparticles of 200 nm (CPC200, Izon Science).

TEM grid micropatterning. The working volume utilized for an individual grid was 20 μL, which was kept constant for the different solutions added onto the grids. Various TEM grids were used in this investigation, including Formvar/Carbon 75 mesh, Copper (Ted Pella, cat #01802-F), Carbon Type-B, 200 mesh TH, Nickel (Ted Pella, cat #01808N), Carbon Type-B, 300 mesh, Nickel (Ted Pella, cat #01813N). The TEM grids were primed with 70% (v/v) ethanol and quickly transferred to PBS via three washes. The grids were then transferred to a 100 μg/mL solution of poly-L-lysine-graft-poly(ethylene glycol) (PLL-g-PEG) and incubated for 1 hour. The excess PLL-g-PEG was washed away by transferring the functionalized grid to PBS three times. After that, PLPP, a commercial photoactivator (Alvéole), was transferred onto the functionalized grid. The grid was then subjected to UV-induced degradation of the PLL-g-PEG by the PRIMO micropatterning system. After micropatterning, the grids were transferred to PBS and washed three times to remove the remaining PLPP.

Electrostatic capture of extracellular particles. A 0.1% (w/v) poly-L-lysine (PLL) solution was incubated on the TEM grid post-micropatterning for 5 min to allow its adsorption onto the micropatterned regions. The grids were then transferred to PBS and washed three times. The extracellular vesicle solution was then incubated onto the functionalized TEM grid for 1 min whereby the unbound extracellular vesicles were subsequently washed off with PBS by pipetting 10 times in an up-and-down motion. The rinsing step was repeated 10 times.

Gold nanoparticle immobilization. A solution of 50-nm streptavidin-functionalized gold nanoparticles (Strep-AuNPs) at various concentrations, including 0.25% and 0.5% (w/v) (GNA50, Nanocs Inc.), was incubated onto the micropatterned TEM grid for 15 min. The grids were then washed with PBS by pipetting PBS 10 times in an up-and-down motion. This rinsing step was repeated 10 times.

Immunogold capture of extracellular particles. TEM grids functionalized with streptavidin-functionalized gold nanoparticles were blocked with a 3% (w/v) of bovine serum albumin (BSA) solution (Sigma-Aldrich) diluted in PBS for 15 min to reduce non-specific capture. Capture antibodies (provided below in Table 1) were biotinylated using the EZ-Link micro Sulfo-NHS-biotinylation kit from (Thermo Fisher Scientific). The biotinylated antibodies were then diluted to 10 μg/mL in a 1% BSA solution and added to the grids for 15 min. Excess antibodies were rinsed away by pipetting PBS 10 times in an up-and-down motion. The rinsing step was repeated 10 times. The surface was further blocked with a 3% (w/v) solution of BSA for 15 min. After removing the BSA solution, murine leukemia virus tagged with V5 (MLV-V5, ViroFlow Technologies) or extracellular vesicles were subsequently incubated onto the grids for 40 min at room temperature in a humid environment. The unbound virions and extracellular vesicles were washed away by pipetting PBS 10 times in an up-and-down motion. The rinsing step was repeated 10 times.

TABLE 1
List of antibodies.

| Antibody | | | Catalog No. | Supplier |
|---|---|---|---|---|
| Capture | EVs | Anti-CD63 | MAB5048 | R&D Systems |
| | | Anti-CD9 | MAB1880 | R&D Systems |
| | MLV-V5 | Anti-V5 tag | AB9116 | Abcam |
| Detection | EVs | Anti-CD63 | SC-5275 | Santa Cruz |
| | | (Alexa Fluor ® | AD488 | Biotechnology |
| | | 488) | | |
| | MLV-V5 | Anti-V5-FITC | R963-25 | Thermo Fisher |
| | | | | Scientific |

Super-resolution microscopy image acquisition. Total internal reflection fluorescence microscopy (TIRFM; Nikon, Melville, NY) was utilized to acquire super-resolution microscopy images. The functionalized TEM grids were placed onto a coverslip and imaged via total internal reflection fluorescence microscopy with a 100× objective and immersion oil to reduce surface refraction. The functionalized TEM grids were maintained in PBS throughout the total internal reflection fluorescence microscopy imaging process.

Negative staining. Two 20 μL droplets of water for injection (WFI) and two 20 μL droplets of negative stain (UranyLess EM stain, Electron Microscopy Sciences) were placed onto parafilm. The functionalized TEM grids were then immersed successively into the two WFI droplets and carefully blotted to remove excess liquid. The grids were then stained via submersion into a droplet of the negative stain, blotted, submersed into the second droplet for ~22 s, and then gently wicked away using filter paper. The stained TEM grids were stored in a grid box overnight to ensure thorough drying.

Electron microscopy image acquisition. TEM brightfield imaging was used at all magnifications. A low magnification range mode at 300× was utilized to locate the center and image the micropatterns. A high magnification range over the selected area at an aperture mode of 15500× and spot size 3 was used for enhanced image quality to view the lower-contrast extracellular particles. Serial acquisition was performed with the SerialEM software (Nexperion, Austria) for a 10×10 array of images. A montage was generated with an Orius SC200 camera via binning 1, pixel size 1.84 nm, number of pieces in X×Y=10×10, piece size in X×Y=2048×2048 pixels, overlap in X×Y=206×206 pixels, and a total area of 34.3×34.3 μm.

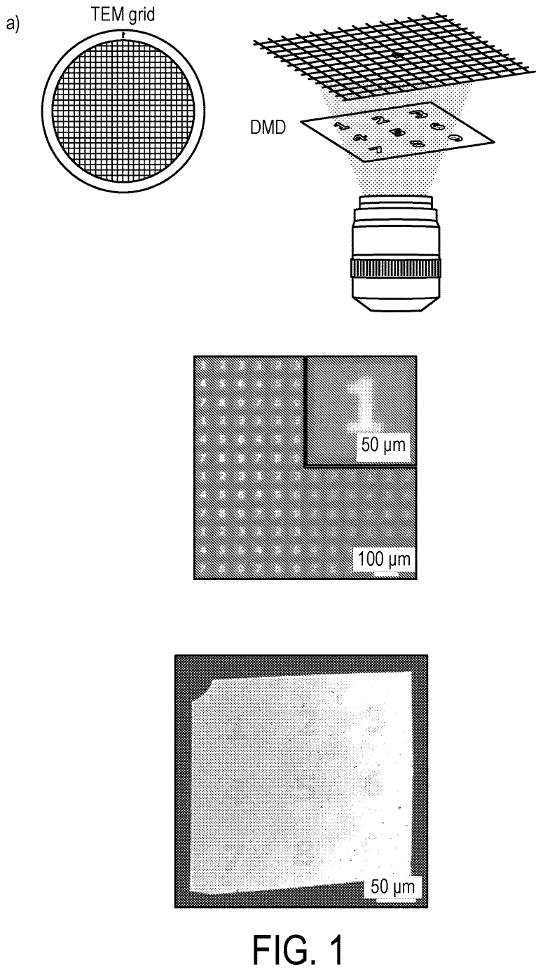

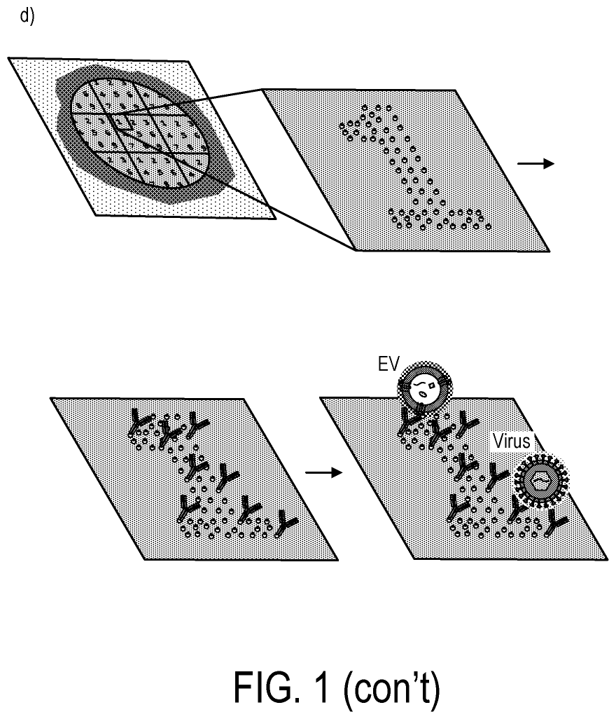

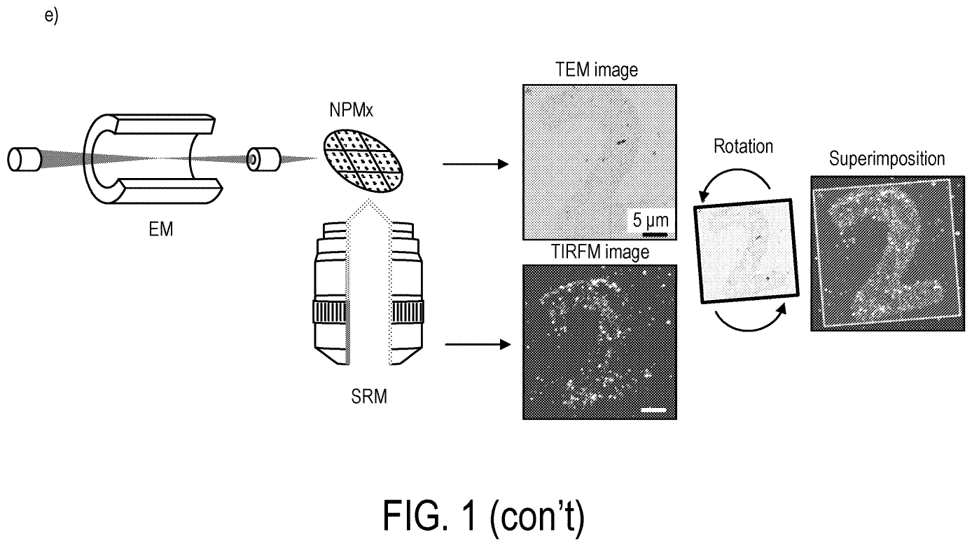

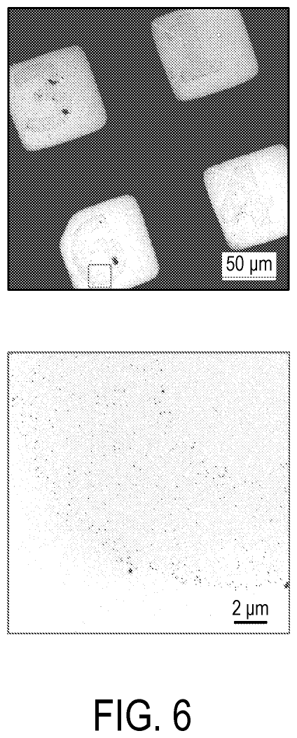

Results. Micropatterning was used to immobilize extracellular particles onto TEM grids to enhance the probability of extracellular particles on the TEM-mesh surface. Briefly, the TEM grids were functionalized with PLL-g-PEG, which forms a brush on the surface and inhibits the adsorption of protein species. After that, the polymer brush was photoetched in specific coordinates utilizing a digital-micromirror device (DMD)-based ultraviolet (UV) projection system to induce the adsorption of protein species (FIG. 1, panel a) [23]. Asymmetric micropatterns, in the present case single-digit numerals, were utilized to determine the orientation of the micropatterns and facilitate superimposition of microscopic techniques (FIG. 1, panel b). The DMD-based micropatterning further enabled the tunability of micropatterns insofar as single-digit or multi-digit arrays in the mesh surface for the adsorption of proteins or protein-conjugated nanoparticles (FIG. 1, panel c; FIG. 6). Immunogold labeling is riddled with bottlenecks, including the stochasticity of particle collision in a colloid mixture due to Brownian motion and the low efficiencies of labeled extracellular particles on the mesh surface post-sample preparation [17, 24]. To address these bottlenecks, the study immobilized streptavidin-functionalized gold nanoparticles onto the micropatterns for their subsequent functionalization with antibodies targeting epitopes on extracellular particles (FIG. 1, panel d). The strategy thus eliminates the Brownian motion of a particle subset in the colloid mixture, in this case the streptavidin-functionalized gold nanoparticles, improving capture and immobilizing the extracellular particles onto the mesh surface and increasing the probability of a labeled extracellular particle in the field of view. Given the distinct features of the asymmetric micropattern that super-resolution microscopy and electron microscopy visualize, the images can be easily superimposed to correlate fluorescent signals to labeled extracellular particles on the mesh surface (FIG. 1, panel e). The term “nano-positioning matrix technique” as described in this example refers to the technique of superimposing super-resolution microscopy and electron microscopy images.

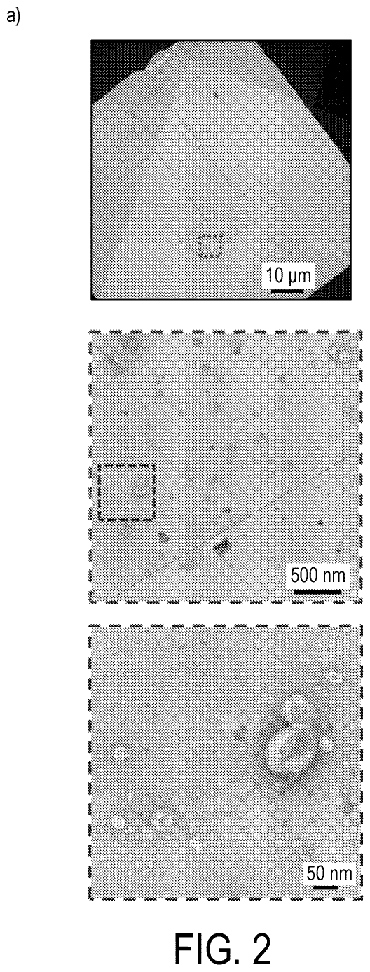

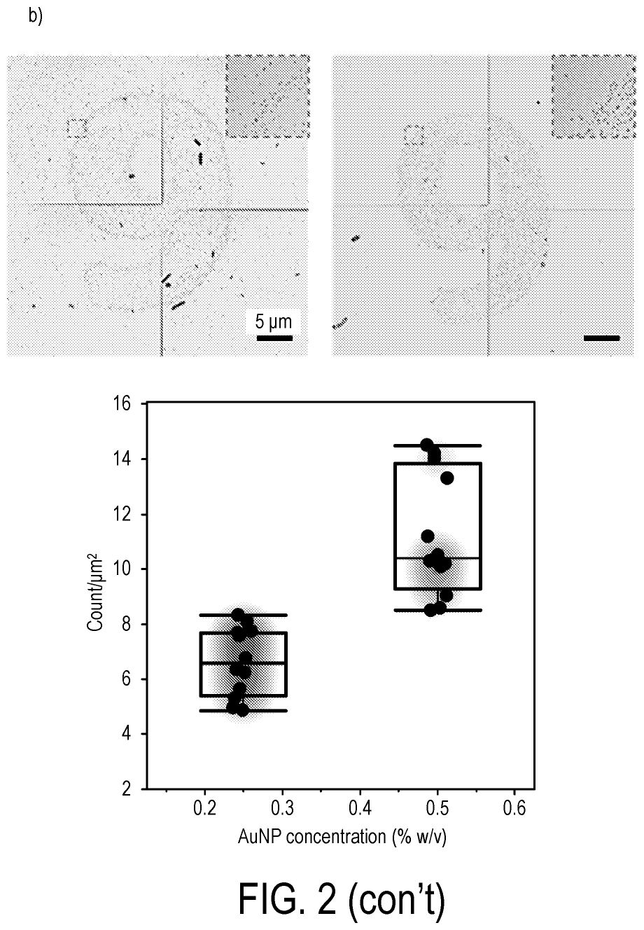

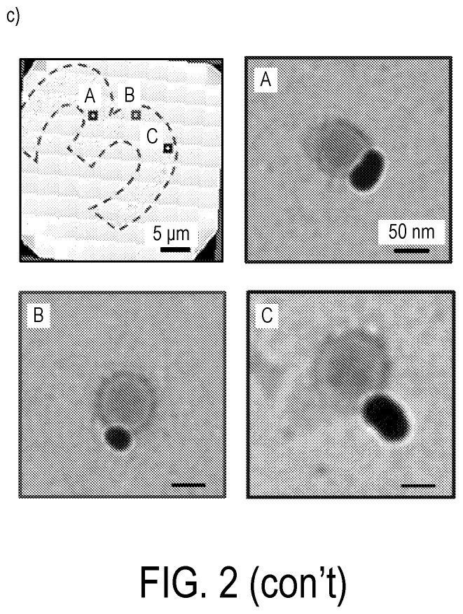

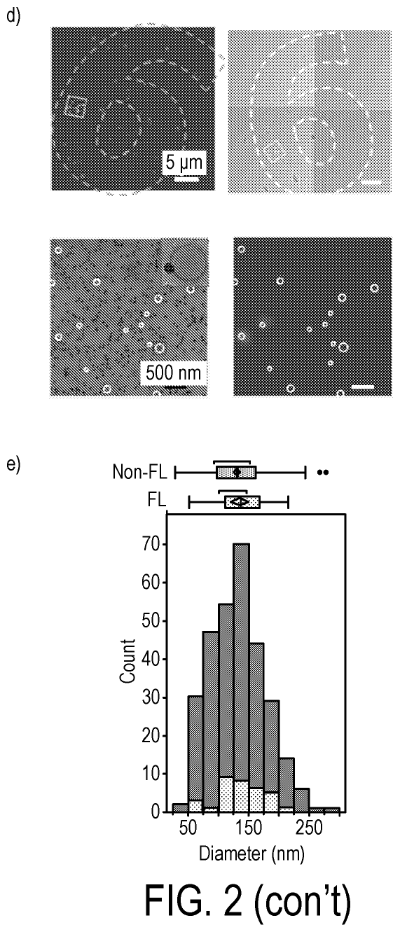

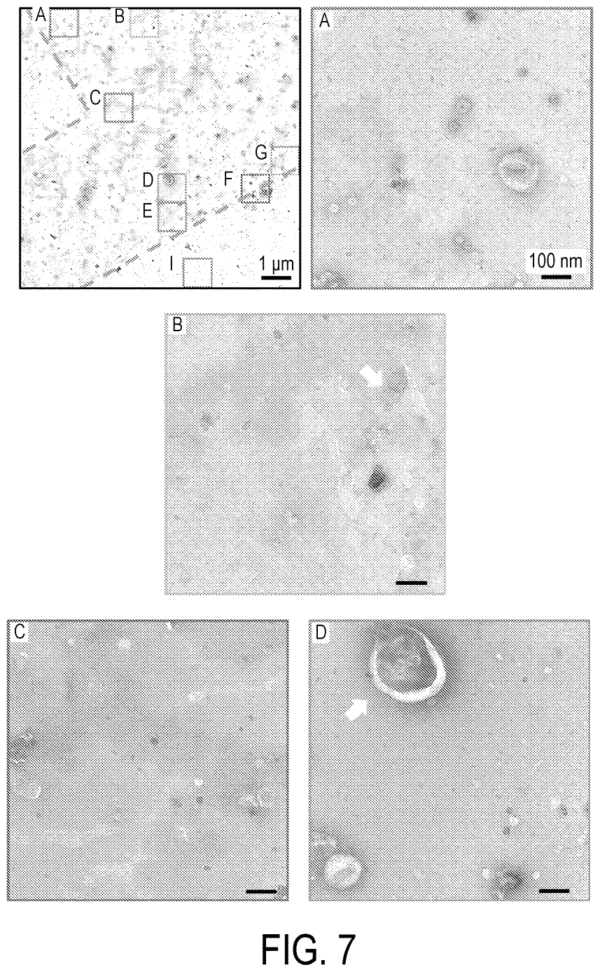

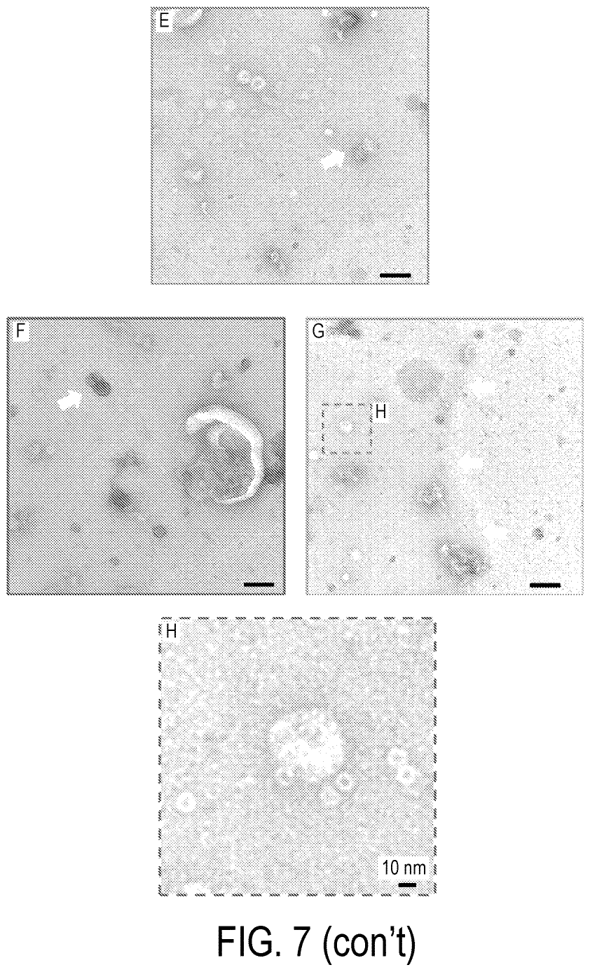

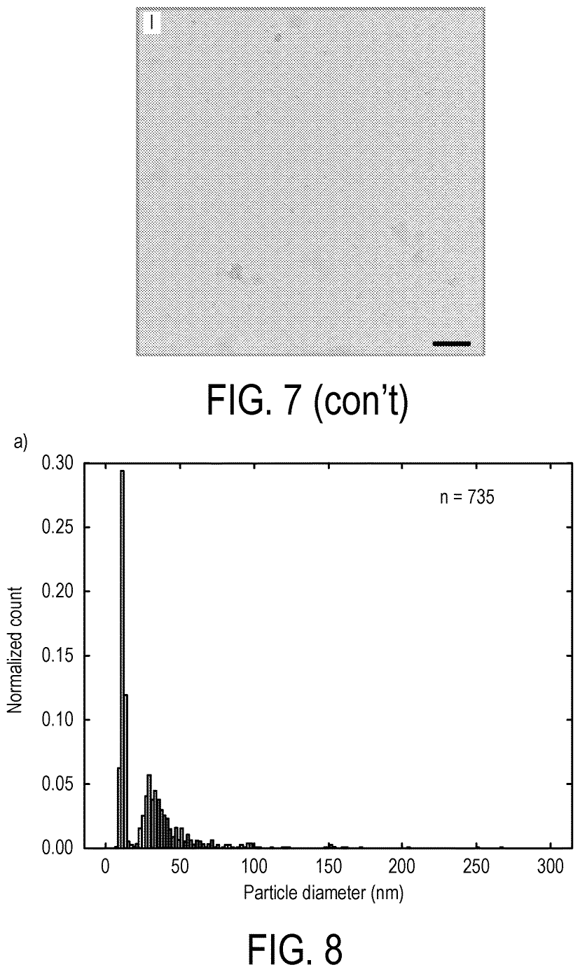

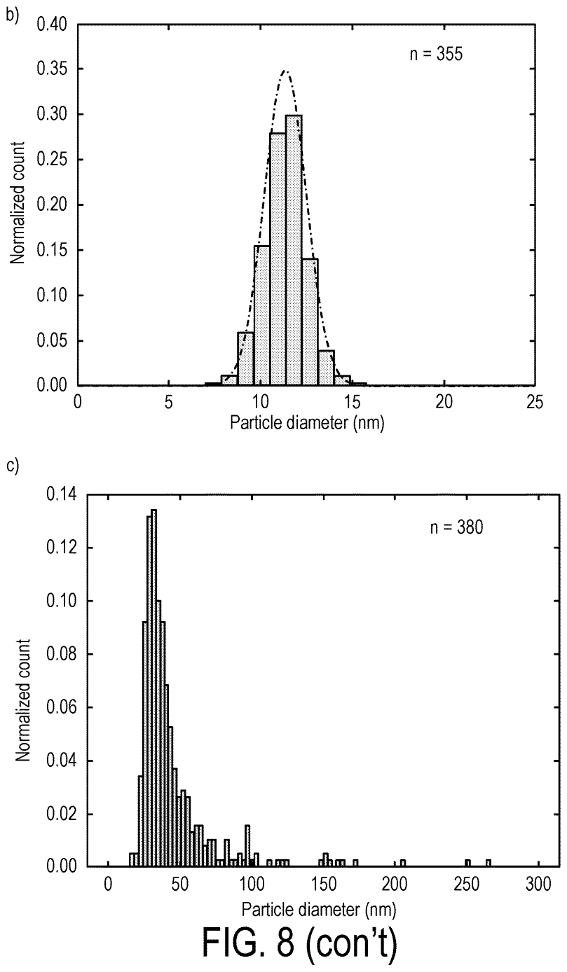

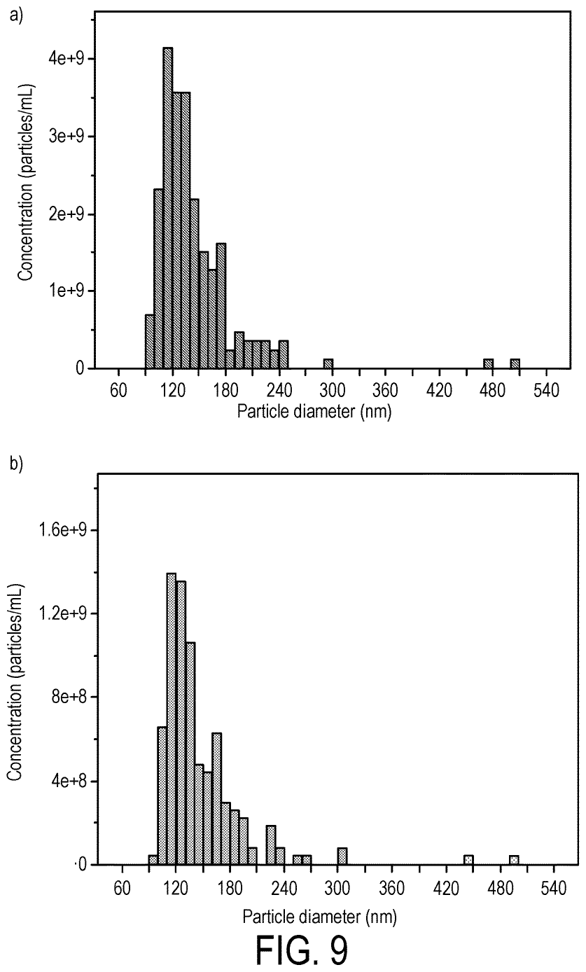

To test the feasibility of the micropatterning method to tether extracellular particles onto the mesh surface, extracellular particles were directed towards the micropatterns via electrostatic interactions to induce the capture of all extracellular particles without capture biases. Therefore, poly-L-lysine, a polycation, was adsorbed onto the photoetched regions to attract negatively charged extracellular vesicles. SEC-purified extracellular vesicles from a glioma cell line bioreactor culture [20,21] at a concentration of ~2×10¹⁰ extracellular vesicles/mL were added to the poly-L-lysine surface demonstrating the capture of a heterogeneous population of extracellular vesicles strictly onto the micropatterned region (FIG. 2, panel a). To scan the entire mesh surface and visualize the numeral, a 10×10 array of TEM images were montaged via SerialEM, a software enabling automation of large-scale electron microscopy acquisition [25]. Within the subpopulation of extracellular vesicles, various morphologies were observed including, classical “cup-shaped” extracellular vesicles, electron-dense extracellular vesicles, multicomponent extracellular vesicles, membrane-compromised extracellular vesicles, and minuscule extracellular particle structures (FIG. 7). The size distribution revealed a bimodal distribution, including an ~11-nm sized normally distributed extracellular particle and a small extracellular vesicle subpopulation (FIG. 8, panels a-c). Interestingly, tunable resistive pulse sensing measurements obscured the bimodal distribution due to the size range falling below the lower limit of detection (FIG. 9, panels a-b).

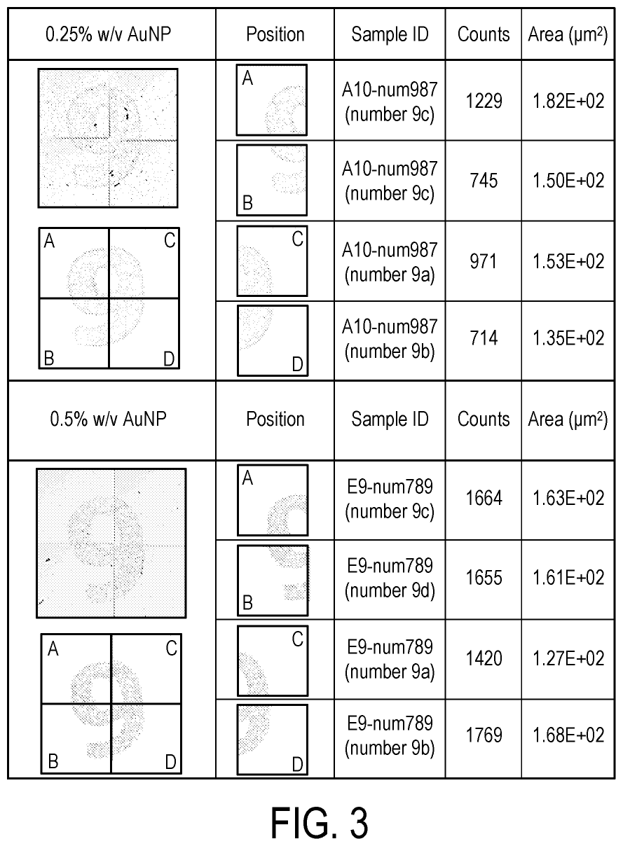

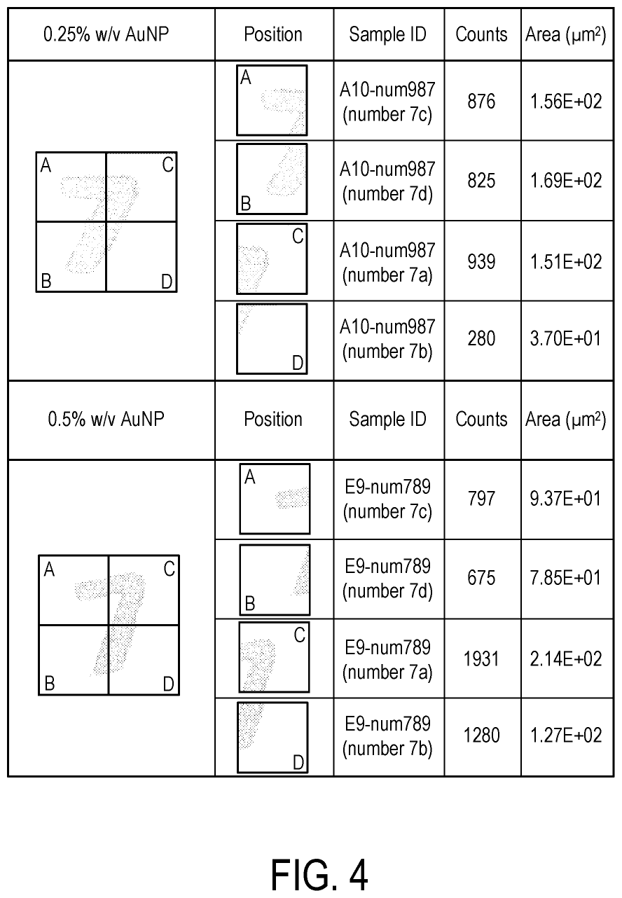

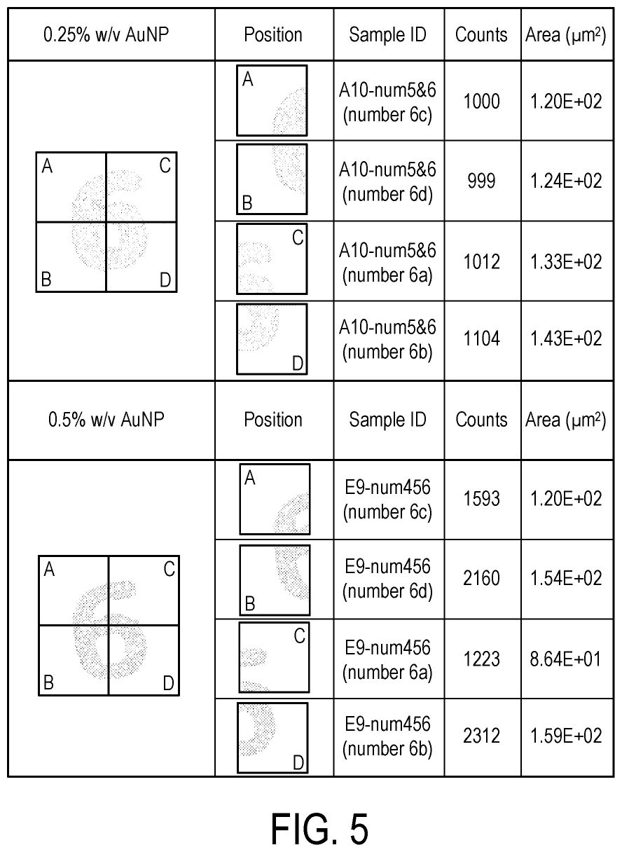

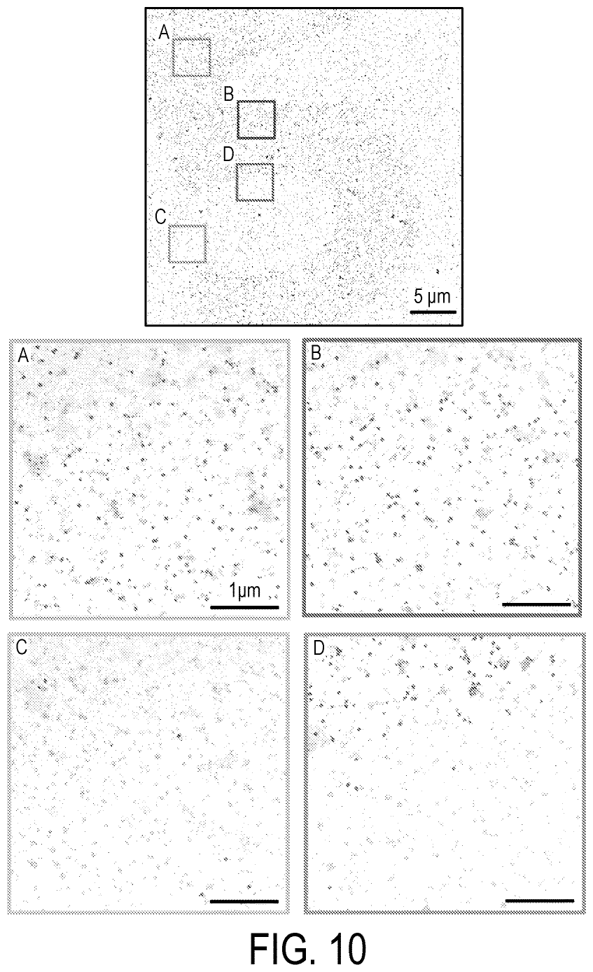

To improve immunogold labeling and demonstrate the nano-positioning matrix technique, 0.25% (w/v) and 0.50% (w/v) of streptavidin-functionalized gold nanoparticles were added to the photoetched regions. Similar to the two-fold increase in concentration, the streptavidin-functionalized gold nanoparticles adsorbed onto the surface normalized by the surface area increased from 6.62±1.24 to 11.20±2.22 (Student's one-tailed t-test, p<0.0001), indicating a concentration-dependent adsorption of the nanoparticles to the mesh surface (FIG. 2, panel b; FIGS. 7-9). Quantification of the streptavidin-functionalized gold nanoparticle adsorption for several characters are shown in FIGS. 3-5. To test immunogold labeling, the streptavidin-functionalized gold nanoparticles were functionalized with antibodies targeting a V5 tag, which is a short peptide sequence frequented in protein purification [26]. MLV virions engineered to express the V5 tag were incubated on the surface at a concentration of ~7×10⁹ virions/mL. As suspected, the MLV virions were observed in proximity to the functionalized gold nanoparticles with little non-specificity outside the micropattern region (FIG. 2, panel c). In the absence of an antibody, the extracellular particles were unable to bind to the micropatterned surface, thus ensuring that binding events were a result of antibody-epitope interactions (FIG. 10).

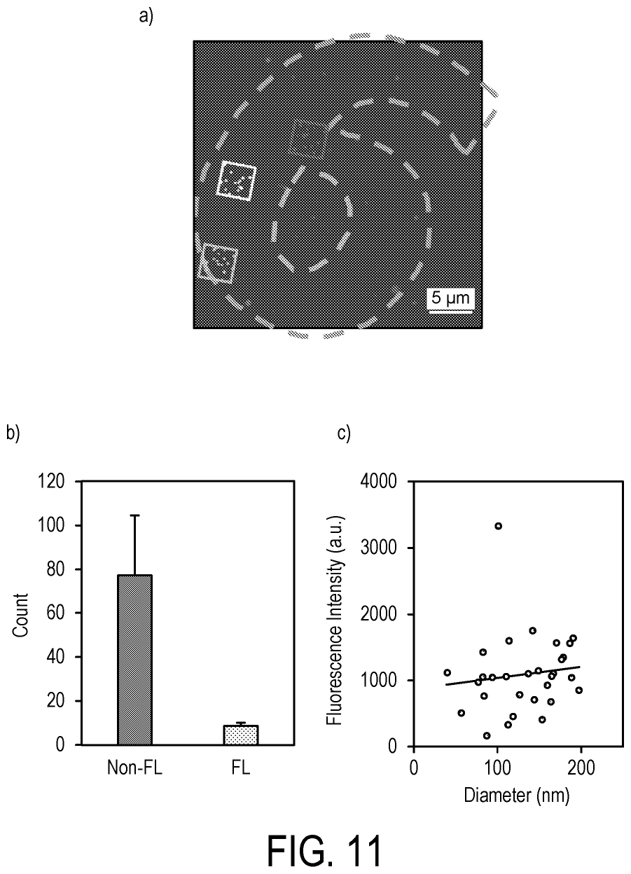

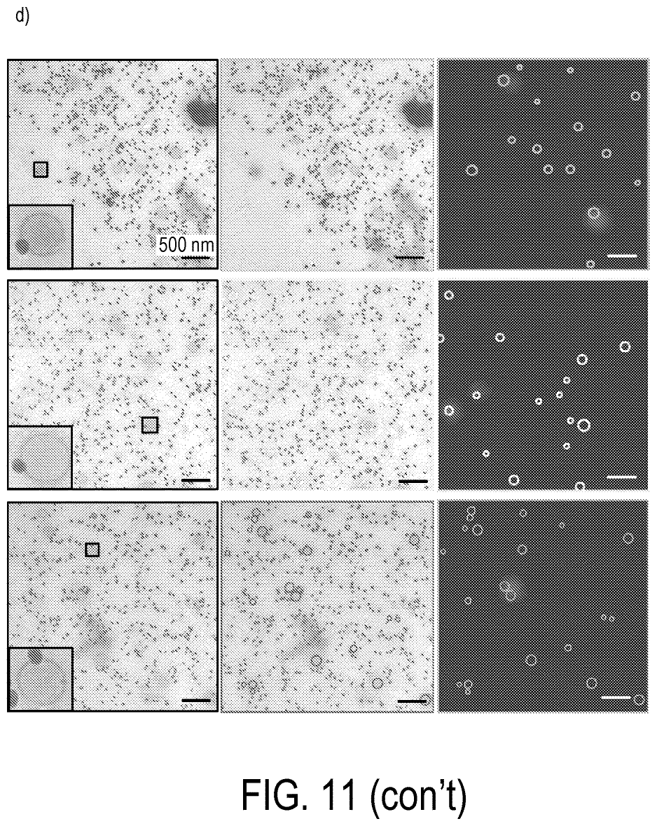

Having a workflow that enabled the immunogold immobilization of extracellular particles in traceable surface coordinates, the nano-positioning matrix technique was utilized to image the same grid via super-resolution microscopy and electron microscopy in an effort to map the micropatterns from one modality to the other. Therefore, extracellular vesicles engineered to fluoresce via the cellular transfection of PalmtdTomato [18,19] were added to streptavidin-functionalized gold nanoparticles functionalized with antibodies targeting CD63. total internal reflection fluorescence microscopy images demonstrated the enrichment of fluorescent signals originating from the micropatterns and forming distinguishable numerals, indicating the capture of extracellular vesicles within the micropattern with minimal non-specificity outside the micropatterned region. Similarly, electron microscopy images yielded distinct numerals via the uniform distribution of gold nanoparticles across the micropatterned surface with extracellular vesicles near the gold nanoparticles. Therefore, the numerals in super-resolution microscopy and electron microscopy images were rotated to match with an in-house algorithm, allowing for the one-to-one mapping of captured extracellular vesicles to fluorescent signals post-offset correction (FIG. 2, panel d, FIG. 11; panel a). Surprisingly, a majority of the extracellular vesicles were not fluorescent (Welch's one-tailed t-test, p=0.0433), indicating that the staining capacity may be less efficient than anticipated (FIG. 11, panel b). Furthermore, the size of the extracellular vesicles by TEM quantification only slightly albeit insignificantly correlated with the fluorescence intensity of the extracellular vesicles by total internal reflection fluorescence microscopy quantification (Pearson's correlation coefficient, p=0.52 for r=0.12), indicating that fluorescence intensities from total internal reflection fluorescence microscopy images are likely due to expression and not size (FIG. 11, panel c). TEM images further revealed that single fluorescent signals may originate from two neighboring extracellular vesicles (FIG. 11, panel d), highlighting the limitations of diffraction-limited techniques, such as total internal reflection fluorescence microscopy, and underscoring the importance of the nano-positioning matrix technique in correlating fluorescent signals to immunogold sorted extracellular vesicles. Although the tunable resistive pulse sensing distributions for the bioreactor-generated extracellular vesicles and the fluorescent extracellular vesicles demonstrated similar sizes, the ~11-nm sized extracellular particle subpopulation was lost and the distribution better resembled the tunable resistive pulse sensing data for both fluorescent and non-fluorescent extracellular vesicles (FIG. 2, panel e), providing insight into the bias of tetraspanin-based capturing.

Discussion and Conclusions. The benefits of using the nano-positioning matrix technique include reducing the required concentration of extracellular particles for electron microscopy, quickening electron microscopy acquisition, and improving immunogold labeling, while providing various modalities, such as the non-biased electrostatic capture of extracellular particles, population-based sorting of extracellular particles, and orthogonal superimposition of optical and non-optical imaging at a single-extracellular vesicle resolution. Current electron microscopy techniques are susceptible to drying-induced artifacts [27] and require high concentrations of extracellular particles for standard electron microscopy and immunogold phenotyping [6]. For patient-derived biofluids, high concentrations of extracellular particles may be a luxury, and reducing concentrations and reproducibility is sought to provide usability across samples. The nano-positioning matrix not only promotes the binding of extracellular particles onto the mesh surface through electrostatics or user-defined immunocapture but also provides the precise coordinates of extracellular particles on the surface, reducing acquisition time, decreasing required concentrations, and its application for high-throughput screening. While nanoparticles are innately subject to Brownian motion, leading to approaches to reduce the stochasticity of movement [6,28-30]. The study circumvented the natural phenomena via the immobilization of one particle species in a colloid mixture thus reducing the dimensionality of particle interactions. The engineered approach enhanced immunogold labeling and provided the precise coordinates of capture to easily superimpose TEM and total internal reflection fluorescence microscopy images and measurements for the first time.

The discussed nano-positioning matrix technique demonstrates the multiparametric characterization of extracellular particles. This technique offers various modalities and combinatorial approaches that enable flexibility in experimental design, allowing for investigations for all types of extracellular particles.

### REFERENCES

1. Jeppesen, D. K., Zhang, Q., Franklin, J. L. & Coffey, R. J. Extracellular vesicles and nanoparticles: emerging complexities. Trends in Cell Biology 33, 667-681 (2023).
2. Hu, S., Hu, Y. & Yan, W. Extracellular vesicle-mediated interorgan communication in metabolic diseases. Trends Endocrinol Metab 34, 571-582 (2023).
3. Gabisonia, K., Khan, M. & Recchia, F. A. Extracellular vesicle-mediated bidirectional communication between heart and other organs. Am J Physiol Heart Circ Physiol 322, H769-H784 (2022).
4. Lucotti, S., Kenific, C. M., Zhang, H. & Lyden, D. Extracellular vesicles and particles impact the systemic landscape of cancer. EMBO J 41, e109288 (2022).

Li, H. et al. Extracellular Vesicular Analysis of Glypican 1 mRNA and Protein for Pancreatic Cancer Diagnosis and Prognosis. Adv Sci (Weinh), e2306373 (2024).
6. Nguyen, L. T. H. et al. An immunogold single extracellular vesicular RNA and protein (J Extracell Vesicles 11, e12258 (2022).
7. Liang, Y., Lehrich, B. M., Zheng, S. & Lu, M. Emerging methods in biomarker identification for extracellular vesicle-based liquid biopsy. J Extracell Vesicles 10, e12090 (2021).
8. Tian, Y. et al. Quality and efficiency assessment of six extracellular vesicle isolation methods by nano-flow cytometry. J Extracell Vesicles 9, 1697028 (2020).
9. Yuana, Y., Levels, J., Grootemaat, A., Sturk, A. & Nieuwland, R. Co-isolation of extracellular vesicles and high-density lipoproteins using density gradient ultracentrifugation. J Extracell Vesicles 3 (2014).
10. Brennan, K. et al. A comparison of methods for the isolation and separation of extracellular vesicles from protein and lipid particles in human serum. Scientific Reports 10, 1039 (2020).
11. Théry, C. et al. Minimal information for studies of extracellular vesicles 2018 (MISEV2018): a position statement of the International Society for Extracellular Vesicles and update of the MISEV2014 guidelines. J Extracell Vesicles 7, 1535750 (2018).
12. Lozano-Andrés, E. et al. Physical association of low density lipoprotein particles and extracellular vesicles unveiled by single particle analysis. J Extracell Vesicles 12, e12376 (2023).
13. Busatto, S. et al. Considerations for extracellular vesicle and lipoprotein interactions in cell culture assays. J Extracell Vesicles 11, e12202 (2022).
14. Sódar, B. W. et al. Low-density lipoprotein mimics blood plasma-derived exosomes and microvesicles during isolation and detection. Sci Rep 6, 24316 (2016).
15. Zhang, J. et al. Engineering a tunable micropattern-array assay to sort single extracellular vesicles and particles to detect RNA and protein in situ. J Extracell Vesicles 12, e12369 (2023).
16. Kwon, Y. & Park, J. Methods to analyze extracellular vesicles at single particle level. Micro and Nano Systems Letters 10, 14 (2022).
17. Erdbrügger, U. & Lannigan, J. Analytical challenges of extracellular vesicle detection: A comparison of different techniques. Cytometry A 89, 123-134 (2016).
18. Lai, C. P. et al. Visualization and tracking of tumour extracellular vesicle delivery and RNA translation using multiplexed reporters. Nat Commun 6, 7029 (2015).
19. Reátegui, E. et al. Engineered nanointerfaces for microfluidic isolation and molecular profiling of tumor-specific extracellular vesicles. Nat Commun 9, 175 (2018).
20. Artuyants, A. et al. Production of Extracellular Vesicles Using a CELLine Adherent Bioreactor Flask. Methods Mol Biol 2436, 183-192 (2022).
21. Hisey, C. L. et al. Investigating the consistency of extracellular vesicle production from breast cancer subtypes using CELLine adherent bioreactors. Journal of Extracellular Biology 1, e60 (2022).
22. Toro-Nahuelpan, M. et al. Tailoring cryo-electron microscopy grids by photo-micropatterning for in-cell structural studies. Nat Methods 17, 50-54 (2020).
23. Strale, P. O. et al. Multiprotein Printing by Light-Induced Molecular Adsorption. Adv Mater 28, 2024-2029 (2016).
24. Park, K., Simmons, S. R. & Albrecht, R. M. Surface characterization of biomaterials by immunogold staining—quantitative analysis. Scanning Microsc 1, 339-350 (1987).
25. Mastronarde, D. N. Automated electron microscope tomography using robust prediction of specimen movements. J Struct Biol 152, 36-51 (2005).
26. Kimple, M. E., Brill, A. L. & Pasker, R. L. Overview of affinity tags for protein purification. Curr Protoc Protein Sci 73, 9.9.1-9.9.23 (2013).
27. Michen, B. et al. Avoiding drying-artifacts in transmission electron microscopy: Characterizing the size and colloidal state of nanoparticles. Sci Rep 5, 9793 (2015).

28 Cohen, A. E. & Moerner, W. E. Suppressing Brownian motion of individual biomolecules in solution. Proc Natl Acad Sci USA 103, 4362-4365 (2006).
29. Hong, C. & Ndukaife, J. C. Scalable trapping of single nanosized extracellular vesicles using plasmonics. Nat Commun 14, 4801 (2023).
30. Yang, Y. et al. Interferometric plasmonic imaging and detection of single exosomes. Proceedings of the National Academy of Sciences 115, 10275-10280 (2018).

The materials and methods of the appended claims are not limited in scope by the specific materials and methods described herein, which are intended as illustrations of a few aspects of the claims and any materials and methods that are functionally equivalent are within the scope of this disclosure. Various modifications of the materials and methods in addition to those shown and described herein are intended to fall within the scope of the appended claims. Further, while only certain representative materials, methods, and aspects of these materials and methods are specifically described, other materials and methods and combinations of various features of the materials and methods are intended to fall within the scope of the appended claims, even if not specifically recited. Thus a combination of steps, elements, components, or constituents can be explicitly mentioned herein; however, all other combinations of steps, elements, components, and constituents are included, even though not explicitly stated.

1. A method for multiparametric characterization of extracellular particles (EPs), the method comprising:
contacting a sample comprising a population of EPs with a micropatterned substrate to adsorb a subpopulation of the EPs on an imaging surface of the substrate, wherein the micropatterned substrate defines an alignment feature disposed thereon,
obtaining an optical image of the adsorbed and/or bound EPs with the alignment feature;
obtaining a non-optical image of the adsorbed and/or bound EPs with the alignment feature; and
correlating the optical image and the non-optical image using the alignment feature as a reference to form a consolidated feature set from the optical and non-optical images.

2. The method of claim 1, further comprising determining structural and/or molecular properties of an individual EP from the consolidated feature set.

3. The method of claim 1, wherein the micropatterned substrate comprises a transmission electron microscope (TEM) grid.

4. The method of claim 1, wherein the micropatterned substrate comprises a functionalized nanolayer disposed on the imaging surface.

5. The method of claim 4, wherein the functionalized nanolayer comprises an extracellular particle binding region, wherein the EP binding region defines a geometry of the alignment feature.

6. The method of claim 5, wherein the functionalized nanolayer comprises a passivation region at least partially surrounding the EP binding region.

7. The method of claim 5, wherein the EP binding region of the functionalized nanolayer comprises an EP capture moiety coupled to a labeling group, wherein the EP capture moiety selectively binds the subpopulation of EPs, and wherein the labeling group is immobilized on the imaging surface.

8. The method of claim 7, wherein the EP capture moiety comprises an antibody, a polypeptide, a nucleic acid, and/or an aptamer.

9. The method of claim 8, wherein the antibody comprises anti-CD63, anti-CD9, anti-V5, conformation-specific antibodies, and/or as a user-defined antibody or antibody cocktail to target EPs.

10. The method of claim 8, wherein the polypeptide comprises a recombinant protein such as ACE2, RGD peptide sequences, and/or electrostatic proteins, such as poly-L-lysine.

11. The method of claim 7, wherein the labeling group comprises a streptavidin-functionalized gold nanoparticle immobilized on the imaging surface by adsorption.

12. The method of claim 6, wherein the passivation region comprises a polyether, polyethylene glycol, and/or poly(L-lysine)-grafted-poly(ethylene glycol) (PLL-g-PEG).

13. The method of claim 4, wherein the alignment feature is etched into the functionalized nanolayer.

14. The method of claim 1, wherein the alignment feature comprises an asymmetric character.

15. The method of claim 1, wherein a concentration of the extracellular particles in the sample is 1×10¹⁴ extracellular particles/mL or less, such as 5×10¹³ extracellular particles/mL or less, 2×10¹³ extracellular particles/mL or less, 5×10¹² extracellular particles/mL or less, 2×10¹² extracellular particles/mL or less, 5×10¹¹ extracellular particles/mL or less, 2×10¹¹ extracellular particles/mL or less, 5×10¹⁰ extracellular particles/mL or less, 2×10¹⁰ extracellular particles/mL or less, 5×10⁹ extracellular particles/mL or less, or 2×10⁹ extracellular particles/mL or less.

16. The method of claim 1, wherein the EPs are tagged with a labeling group.

17. The method of claim 1, wherein the EPs are tagged with a fluorophore, dye, or fluorescent protein, such as a fluorescent-conjugated antibody and/or chromophore.

18. The method of claim 1, wherein the step of correlating the optical and non-optical images comprises forming a composite image by rotating and/or translating one or more of the optical and/or non-optical images to superimpose the alignment feature between the optical and non-optical images.

19. The method of claim 1, wherein the optical image comprises a super-resolution microscopy (SRM) image.

20. The method of claim 19, wherein the SRM image comprises a total internal reflection fluorescence microscope (TIRFM) image.

21. The method of claim 1, wherein the non-optical image comprises an electron microscope (EM) image.

22. The method of claim 21, wherein the EM image comprises a TEM image.

23. A system for the multiparametric characterization of extracellular particles (EPs), the system comprising:
a micropatterned substrate having an imaging surface and defining an alignment feature disposed thereon, wherein the micropatterned substrate is structured to adsorb a subpopulation of EPs on the imaging surface from a sample,
an optical imaging element positionable to collect optical images of adsorbed EP and the alignment feature;
a non-optical imaging element positionable to collect non-optical images of adsorbed and/or bound EPs and the alignment feature; and
a processor; and
a memory having instructions stored thereon, wherein execution of the instructions by the processor causes the processor to:
receive optical and non-optical images showing the subpopulation of adsorbed and/or bound EPs; and
orient the optical and non-optical images using the alignment feature to form a consolidated feature set from the optical and non-optical images.

24. The system of claim 23, wherein the processor is further configured to determine structural and/or molecular properties of an individual EP from the consolidated feature set.

25. The system of claim 23, wherein the micropatterned substrate comprises a transmission electron microscope (TEM) grid.

26. The system of claim 23, wherein the micropatterned substrate comprises a functionalized nanolayer disposed on the imaging surface.

27. The system of claim 26, wherein the functionalized nanolayer comprises an extracellular particle binding region, wherein the EP binding region defines a geometry of the alignment feature.

28. The system of claim 27, wherein the functionalized nanolayer comprises a passivation region at least partially surrounding the EP binding region.

29. The system of claim 27, wherein the EP binding region of the functionalized nanolayer comprises an EP capture moiety coupled to a labeling group, wherein the EP capture moiety selectively binds the subpopulation of EPs, and wherein the labeling group is immobilized on the imaging surface.

30. The system of claim 29, wherein the EP capture moiety comprises an antibody, a polypeptide, a nucleic acid, and/or an aptamer.

31. The system of claim 30, wherein the antibody comprises anti-CD63, anti-CD9, and/or anti-V5 conformation-specific antibodies, and/or as a user-defined antibody or antibody cocktail to target EPs.

32. The system of claim 30, wherein the polypeptide comprises a recombinant protein such as ACE2, RGD peptide sequences, and/or electrostatic proteins, such as poly-L-lysine.

33. The system of claim 29, wherein the labeling group comprises a streptavidin-functionalized gold nanoparticle immobilized on the imaging surface by adsorption.

34. The system of claim 28, wherein the passivation layer comprises polyether, polyethylene glycol, and/or poly(L-lysine)-grafted-poly(ethylene glycol) (PLL-g-PEG).

35. The system of claim 26, wherein the alignment feature is etched into the functionalized nanolayer.

36. The system of claim 23, wherein the alignment feature comprises an asymmetric character.

37. The system of claim 23, wherein the the EPs are tagged with a labeling group.

38. The system of claim 23, wherein the EPs are tagged with a fluorophore, dye, or fluorescent protein, such as a fluorescent-conjugated antibody and/or chromophore.

39. The system of claim 23, wherein the processor is configured to generate a composite image of the optical and non-optical images by rotating and/or translating one or more of the images to superimpose the alignment feature in the optical and non-optical images.

40. The system of claim 23, wherein the optical image comprises a super-resolution microscopy (SRM) image.

41. The system of claim 40, wherein the SRM image comprises a total internal reflection fluorescence microscope (TIRFM) image.

42. The system of claim 23, wherein the non-optical image comprises an electron microscope (EM) image.

43. The system of claim 42, wherein the EM image comprises a TEM image.
